(19) United States
(12) Patent Application Publication
(10) Pub. No.: US 2026/0248092 A1
(43) Pub. Date: Aug. 27, 2026

(54) SORGHUM MATERNAL HAPLOID INDUCING LINES AND METHODS OF PRODUCING DOUBLED HAPLOIDS

(71) Applicant: PIONEER HI-BRED INTERNATIONAL, INC., JOHNSTON, IA (US)

(72) Inventors: SREEKALA CHELLAMMA, WAUKEE, IA (US); NATHAN ALLEN DAVIS, NEWTON, IA (US); TARA LUANA BARRETT TARNOWSKI, DES MOINES, IA (US); MARIA LAURA MAYOR, MANHATTAN, KS (US); REBEKAH ANNE COURTER-CRISPIN, JOHNSTON, IA (US); STEPHEN MICHAEL SCHRADER, ANKENY, IA (US)

(73) Assignee: PIONEER HI-BRED INTERNATIONAL, INC., (omitted)

(21) Appl. No.: 19/545,431

(22) Filed: Feb. 20, 2026

Related U.S. Application Data
(60) Provisional application No. 63/761,345, filed on Feb. 21, 2025.

(51) Int. Cl. A01H 6/46; A01H 3/04; A01H 5/10

(52) U.S. Cl. CPC A01H 6/4666; A01H 3/04; A01H 5/10

(57) ABSTRACT

Three sorghum maternal haploid inducer lines 2PWCS25R, 2PNUM88R, and 2PCNW79R are provided including seed, plants and plant parts thereof. Methods for producing sorghum haploid embryos using any of 2PWCS25R, 2PNUM88R, or 2PCNW79R are also provided. The sorghum haploid embryos/seeds/plants produced as a result of the use of any of maternal haploid inducer line 2PWCS25R, 2PNUM88R, or 2PCNW79R may be doubled to produce doubled haploid embryos, seeds, and plants (e.g., as part of a sorghum breeding program). Also provided is a method of generating a haploid sorghum embryo comprising performing emasculation on a first sorghum plant. The emasculation comprises contacting the first plant with Trifluoromethanesulfonamide (TFMSA).

### FIELD

The present invention relates to the field of sorghum breeding and doubled haploid production.

### BACKGROUND

Sorghum, Sorghum bicolor L., is diploid with 10 pairs of chromosomes (2n=20). Sorghum is an important and valuable food and feed grain crop. In addition, its vegetative parts are used for forage, syrup and shelter. Thus, a continuing goal of plant breeders is to develop stable high yielding sorghum hybrids that are agronomically sound. The reasons for this goal are to maximize the amount of grain produced on the land used and to supply food for both animals and humans.

However, the development of new inbred and hybrid sorghum plants is slow and costly, lagging behind that of other crops such as maize. One reason is that doubled haploid breeding systems are lacking for sorghum, mainly because there are limited sorghum haploid inducer lines needed to produce sorghum haploid embryos, which is the first step in any doubled haploid production system.

### SUMMARY

Three novel sorghum haploid inducing lines, 2PWCS25R, 2PNUM88R, and 2PCNW79R, are provided herein. Representative seed of the haploid inducing lines 2PWCS25R, 2PNUM88R, and 2PCNW79R has been deposited with the National Center for Marine Algae and Microbiota (NCMA) on Jul. 8, 2024, under accession numbers 202407001, 202407002, and 202407003, respectively. Provided herein are seeds, plants, non-seed plant parts, and cells of sorghum lines 2PWCS25R, 2PNUM88R, and 2PCNW79R. Sorghum haploid embryos or plants produced by crossing a plant of 2PWCS25R, 2PNUM88R, or 2PCNW79R with a second plant, in which the second plant is used as a female, and sorghum haploid plants produced by growing the sorghum haploid embryo or seed, are also provided. Progeny of 2PWCS25R, 2PNUM88R, and 2PCNW79R are also provided.

2PWCS25R, 2PNUM88R, and 2PCNW79R were each produced from F1 crosses between SMHI01 and SMHI02, followed by subsequent F2, F3, and/or backcrosses (see Example 4). SMHI01 and SMHI02 are described in U.S. Pat. Nos. 11,089,748B2 11,737,405B2, both of which are incorporated herein by reference in their entirety.

Methods of producing sorghum haploid embryos or seed are also provided in which the sorghum haploid embryos or seed are produced by pollinating a female sorghum diploid plant with pollen from sorghum haploid inducer lines 2PWCS25R, 2PNUM88R, or 2PCNW79R.

Methods of producing sorghum doubled haploid embryos, seed, or plants are also provided in which a sorghum haploid embryo or seed or a sorghum haploid plant, produced by pollinating female diploid plants with pollen from any one of sorghum haploid inducer lines 2PWCS25R, 2PNUM88R, or 2PCNW79R, is placed in contact with a chromosome doubling agent. In other aspects, a sorghum doubled haploid plant may be produced by growing a sorghum doubled haploid embryo or seed into a sorghum doubled haploid plant.

Also provided are methods for making a plant in which sorghum line 2PWCS25R, 2PNUM88R, or 2PCNW79R is crossed with another plant. A hybrid seed, plant, or plant part produced by crossing line 2PWCS25R, 2PNUM88R, 2PCNW79R, or a locus conversion of any of 2PWCS25R, 2PNUM88R, or 2PCNW79R, with another plant is also provided.

Provided herein is a plant, non-seed plant part, seed, or cell of any one of sorghum varieties 2PWCS25R, 2PNUM88R, or 2PCNW79R. Representative seed of each variety was deposited with the NCMA on Jul. 8, 2024.

Provided herein is a method of producing a sorghum haploid embryo or seed, the method comprising pollinating a female sorghum diploid plant with pollen from sorghum haploid inducer line 2PWCS25R, 2PNUM88R, or 2PCNW79R. The pollination results in production of a sorghum haploid embryo and/or haploid seed.

Provided herein is a method of generating a haploid sorghum embryo. The method comprises performing emasculation on a first sorghum plant. The emasculation comprises contacting the first plant with Trifluoromethanesulfonamide (TFMSA). The method comprises crossing a male gamete derived from a haploid inducer plant with a female sorghum gamete derived from the first sorghum plant, thereby generating the haploid embryo.

### DETAILED DESCRIPTION

### Definitions

In the description and examples that follow, a number of terms are used herein. In order to provide a clear and consistent understanding of the specification and claims, including the scope to be given such terms, the following definitions are provided:

A haploid plant has a genome containing a single set of chromosomes. The reduced number of chromosomes (n) in the haploid plant is equal to that in the gamete (for Sorghum bicolor, n=10).

A diploid plant has a genome containing two sets of chromosomes. The chromosome number (2n) is equal to that in the zygote (for Sorghum bicolor, 2n=20).

A “doubled haploid” or “doubled haploid plant” (or cell) is one that is developed by the doubling of a haploid set of chromosomes. A doubled haploid plant is considered a homozygous plant. A plant is considered to be doubled haploid if it is fertile, even if the entire vegetative part of the plant does not consist of the cells with the doubled set of chromosomes. For example, a plant will be considered a doubled haploid plant if it contains viable gametes, even if it is chimeric.

A “haploid immature embryo” is defined as the embryo formed after one sperm nucleus from a pollen grain fuses with the polar nuclei in the embryo sac to create a triploid (3N) endosperm and before dry down.

A “doubled haploid embryo” is an embryo that has one or more cells that contain 2 sets of homozygous chromosomes.

The phrases “contacting”, “comes in contact with” or “placed in contact with” can be used to mean “direct contact” or “indirect contact”. For example, the medium comprising a chromosome doubling agent may have direct contact with the haploid cell or the medium comprising the chromosome doubling agent may be separated from the haploid cell by filter paper, plant tissues, or other cells. In such cases, the chromosome doubling agent is transferred through the filter paper or cells to the haploid cell.

As used herein, the term “plant” includes reference to whole plants, plant organs (e.g., leaves, stems, roots, etc.), seeds and plant cells and progeny of same. “Plant cell”, as used herein includes, without limitation, seeds, suspension cultures, embryos, meristematic regions, callus tissue, leaves, roots, shoots, gametophytes, sporophytes, pollen, and microspores. The class of plants which can be used in the methods of provided include both monocotyledonous and dicotyledonous plants.

Locus Conversion (Also called a Trait Conversion): A locus conversion refers to a modified plant within a variety that retains the overall genetics of the variety and further includes a locus with one or more specific desired traits, and otherwise has the same physiological and morphological characteristics of the variety, such as those described in the Examples disclosed herein. Traits can be directed to, for example, modified grain, male sterility, insect control, disease control or herbicide tolerance. Traits can be mutant genes, transgenic sequences or native traits. A single locus conversion refers to plants within a variety that have been modified in a manner that retains the overall genetics of the variety and include a single locus with one or more specific desired traits. A single locus conversion can include at least or about 1, 2, 3, 4 or 5 traits and less than or about 15, 10, 9, 8, 7 or 6 traits. A locus converted plant can include, for example, at least or about 1, 2 or 3 and less than or about 20, 15, 10, 9, 8, 7, 6, or 5 modified loci while still retaining the overall genetics of the variety and otherwise having essentially the same, the same, all or essentially all of the physiological and morphological characteristics of the variety. The total number of traits at one or more locus conversions can be, for example, at least or about 1, 2, 3, 4 or 5 and less than or about 25, 20, 15, 10, 9, 8, 7 or 6. Examples of single locus conversions include mutant genes, transgenes and native traits finely mapped to a single locus. Traits may be introduced by transformation, backcrossing, or a combination of both.

“Line” and “Variety” are used synonymously herein to refer to the haploid inducer sorghum plants disclosed herein.

Three maternal haploid inducing lines, 2PWCS25R, 2PNUM88R, and 2PCNW79R have been developed in sorghum (Sorghum bicolor). To validate their utility as maternal haploid inducers, sorghum female plants were pollinated with pollen from the newly developed haploid inducing lines, and seeds were harvested from the female plants. The harvested seed was either screened itself for haploid seed or planted to screen for putative haploid plants. Haploid plants were validated via genetic marker screening, flow cytometry, and/or phenotypic analysis.

### Sorghum lines 2PWCS25R, 2PNUM88R, and 2PCNW79R

Provided herein is a plant, non-seed plant part, seed, or cell of any one of sorghum varieties 2PWCS25R, 2PNUM88R, or 2PCNW79R. Representative seed of each variety has been deposited with the Nation Center for Marine Algae and Microbiota (NCMA) on Jul. 8, 2024. The plants or non-seed plant parts are suitable for the generation of haploid plants via the pollination of female plants or plant parts and selection of resulting haploid seeds, embryos, and/or plants. The newly generated haploid seeds, embryos, and/or plants can then be used to generate doubled haploid plants via chromosome doubling. A sorghum haploid embryo or seed can be produced by crossing a 2PWCS25R, 2PNUM88R, or 2PCNW79R plant with a second plant. The second plant is used as a female. The sorghum haploid embryo or seed can be grown into a haploid plant. It is expected that both F1 hybrids and haploid embryos/seed will be produced, with the haploid embryos/seed comprising genetic material originating from the female second plant.

In some examples, the plant, non-seed plant part, seed, or plant cell further

comprises a locus conversion. The plant or a plant grown from the plant part, seed, or plant cell otherwise comprises all of the physiological and morphological characteristics of sorghum variety 2PWCS25R, 2PNUM88R, or 2PCNW79R when grown under the same environmental conditions as the unmodified variety.

The 2PWCS25R, 2PNUM88R, and 2PCNW79R varieties can both be bred with other sorghum lines to create further varieties. In such examples, production of haploid embryos/seed may not be an objective and, instead, production of hybrid seed can be sought. In some examples, a sorghum seed can be produced by crossing the sorghum variety 2PWCS25R, 2PNUM88R, or 2PCNW79R with a different plant. The seed can be used to produce a plant (e.g., a further sorghum variety). In some examples, the produced plant can be an F1 hybrid plant. In some examples, the F1 hybrid plant can also be evaluated for its ability to induce haploid plants/embryos/seeds when used as a male.

### Methods Using Sorghum Lines 2PWCS25R, 2PNUM88R, and 2PCNW79R

The sorghum varieties disclosed herein can be used to produce haploid embryos/seeds/plants. In general, the sorghum varieties disclosed herein can be used as a male (i.e., as a pollen source) to produce progeny with a female plant or plant part. A portion of the seeds/embryos produced by the cross between the two plants can be haploid, with nuclear chromosomes only derived from the female plant. The haploid seeds, embryos, and/or plants grown therefrom can then be selected (e.g., by marker genotyping and/or flow cytometry). The selected, haploid seeds, embryos, and/or plants can then be induced to undergo chromosome doubling to produce a homozygous, diploid (2n) plant.

Accordingly, provided herein is a method of producing a sorghum haploid embryo or seed. The method comprises pollinating a female sorghum diploid plant with pollen from sorghum haploid inducer line 2PWCS25R, 2PNUM88R, or 2PCNW79R. The pollination results in production of a sorghum haploid embryo and/or haploid seed.

In some examples, the method further comprises, prior to pollinating the female sorghum diploid plant, performing emasculation by contacting the female diploid sorghum plant with Trifluoromethanesulfonamide (TFMSA). Contact with TFMSA can result in a plant that is unable to produce male gametes. Thus, such a contacted plant can be reliably pollinated by the haploid inducer lines of the present disclosure, with reduced or eliminated interference from self-produced pollen. In other examples, alternative emasculation techniques can be used, as described herein (e.g., hot water treatment, clipping of florets, or cytoplasmic male sterility).

In some examples, the method further comprises contacting, with a chromosome doubling agent, any of (1) the sorghum haploid embryo, (2) haploid seed, or (3) a haploid plant germinated from the haploid seed or embryo. The chromosome doubling agent can be any agent suitable to cause a doubling of the chromosomes in a cell of the haploid plant to create a diploid genome in the cell. Colchicine or other doubling agents described herein can be used. Thus, a doubled haploid plant can be produced from the haploid embryo, seed, or plant. The doubled haploid plant (now with a 2n chromosome number) can be grown to produce seed.

Also provided herein is a method of producing a sorghum seed. The method comprises crossing two sorghum plants and harvesting the resultant sorghum seed. At least one of the sorghum plants is a sorghum variety of the present disclosure (2PWCS25R, 2PNUM88R, or 2PCNW79R). In some examples, the produced seed can be grown into an F1 plant and crossed with another sorghum plant to produce another generation of seed.

Also provided herein is a method of plant breeding. The method comprises: (a) crossing a plant or non-seed plant part of any one of the varieties disclosed herein with a second plant comprising a desired single locus or loci to produce F1 progeny plant(s); and (b) selecting at least a first progeny plant from step (a) that comprises the single locus or loci to produce a selected progeny plant.

### Compositions

The three novel sorghum haploid inducing lines 2PWCS25R, 2PNUM88R, and 2PCNW79R are provided herein including seeds, plants, non-seed plant parts, and cells of each of sorghum lines 2PWCS25R, 2PNUM88R, or 2PCNW79R.

Sorghum haploid embryos, seeds, or plants produced by crossing a plant of 2PWCS25R, 2PNUM88R, or 2PCNW79R with a second plant, in which the second plant is used as a female, and sorghum haploid plants produced by growing the sorghum haploid embryo or seed, are also provided.

### Methods of Use Relating to Haploid Induction

Also provided are methods for 1) producing sorghum haploid embryos or seed, 2) producing sorghum doubled haploid embryos, seed, or plants by contacting sorghum haploid embryos, seed, or plants with a chromosome doubling agent, and 3) producing sorghum doubled haploid plants by contacting sorghum haploid embryos or seed with a chromosome doubling agent and growing the sorghum doubled haploid embryos or seed into sorghum doubled haploid plants.

To generate sorghum haploid embryos, seeds, or plants, pollen from any of 2PWCS25R, 2PNUM88R, or 2PCNW79R can be used to pollinate the stigmas of sorghum diploid plants. Prior to pollination, the sorghum plants that are to be used as females may be emasculated using any emasculation technique. In some examples, the plants to be used as females can be subjected to TFMSA treatment as disclosed herein. In other examples, part of the peduncle may be emasculated by clipping off florets that are about to flower; a sorghum head may be contacted with hot water (typically 45-48° C. for a period of about 10 minutes); or sorghum heads may be covered with a plastic bag to create high humidity inside the bag, thereby inhibiting pollen shed. Alternatively, the female plants may have a cytoplasmic male sterility trait. The stigmas of the female parent sorghum plants may then be pollinated with viable pollen grains collected from the anthers of 2PWCS25R, 2PNUM88R, or 2PCNW79R. Pollination may be performed using a paint brush in a controlled environment; the collected pollen may be dusted over exposed stigma; or the pollen producing head (from the haploid male inducer line) may be brushed over an emasculated head. Identification of sorghum haploid embryos may be aided by the detection of genetic markers, plant phenotype, and/or flow cytometry.

Sorghum haploid cells, haploid embryos, haploid seeds, haploid seedlings or haploid plants can be treated with a chromosome doubling agent. Homozygous plants can be regenerated from haploid cells by contacting the haploid cells, such as haploid embryo cells, with chromosome doubling agents. The haploid cells may come in contact with the chromosome doubling agent at the time of pollination, anytime after pollination, typically 6 hours to 21 days after pollination, 6 hours to 15 days after pollination, at the mature seed stage, at the seedling stage, or at the plant stage. The haploid embryo may come in contact with the chromosome doubling agent when the haploid embryo is formed or anytime after pollination, for example, 6 hours to 21 days after pollination, 6 hours to 15 days after pollination, or at the mature seed stage. The haploid embryo may be isolated. It may be contained within the kernel, ovule, or seed. It may also be on the panicle in the case of sorghum . The panicle comprising the haploid embryo may be on the plant or isolated from the plant. The panicle also may be sectioned. After chromosome doubling, the doubled haploid embryo will contain 2 copies of maternally derived chromosomes.

Methods of chromosome doubling are disclosed in Antoine-Michard, S. et al., Plant cell, tissue organ cult., Cordrecht, the Netherlands, Kluwer Academic Publishers, 1997, 48(3): 203-207; Kato, A., Maize Genetics Cooperation Newsletter 1997, 36-37; and Wan, Y. et al., TAG, 1989, 77:889-892. Wan, Y. et al., TAG, 1991, 81:205-211, the disclosures of which are incorporated herein by reference. Typical methods involve contacting the cells with colchicine, anti-microtubule agents or anti-microtubule herbicides, pronamide, nitrous oxide, trifluralin, or any mitotic inhibitor to create homozygous doubled haploid cells. The amount of colchicine used in medium is generally 0.01%-0.2% by weight or approximately 0.05% by weight or APM can be used at 5-225 μM. In some examples, the amount of colchicine can range from approximately 50-600 mg/L such as, for example, approximately 50 mg/L, 100 mg/L, 150 mg/L, 200 mg/L, 250 mg/L, 300 mg/L, 350 mg/L, 400 mg/L, 450 mg/L, or 500 mg/L. The amount of pronamide in medium is approximately 0.5-20 μM. Other agents may be used with the mitotic inhibitors to improve doubling efficiency. Such agents may be dimethyl sulfoxide (DMSO), adjuvants, surfactants, and the like. Examples of mitotic inhibitors are included in Table 1.

TABLE 1
Chemical chromosome doubling agents

| | | |
|---|---|---|
| Common Name/ Trade name | CAS | IUPAC |

Colchicine and Colchicine Derivatives

| | |
|---|---|
| colchicine / | (S)-N-(5,6,7,9-tetrahydro-1,2,3,10- |
| acetyltrimethylcol-chicinic | tetramethoxy-9-oxobenzo (a) |
| acid | heptalen-7-yl) acetamide |
| colchicine derivatives | |

Carbamates

| | | |
|---|---|---|
| Carbetamide | (R)-1-(ethylcarbamoyl)ethyl | (2R)-N-ethyl-2- |
| | carbanilate | [[(phenylamino)carbonyl]oxy]pro- |
| | | panamide |
| chloropropham | | |
| Propham | | |

Benzamides

| | | |
|---|---|---|
| Pronamide / propyzamide | 3,5-dichloro-N-(1,1- | 3,5-dichloro-N-(1,1-dimethyl-2- |
| | dimethylpropynyl)ben-zamide | propynyl)benzamide |
| Tebutam | | |

Benzoic Acids

| | | |
|---|---|---|
| Chlorthal dimethyl | | |
| (DCPA), | | |
| Dicamba / dianat/ | 3,6-dichloro-o-anisic acid | 3,6-dichloro-2-methoxybenzoic acid |
| disugran (dicamba- | | |
| methyl) (BANVEL, | | |
| CLARITY) | | |

Dinitroaniline chromosome doubling agents

| | | |
|---|---|---|
| benfluralin / benefin / | N-butyl-N-ethyl-α,α,α- | N-butyl-N-ethyl-2,6-dinitro-4- |
| (BALAN) | trifluoro-2,6-dinitro-p-toluidine | (trifluoromethyl)benzenamine |
| Butralin | (RS)-N-sec-butyl-4-tert- | 4-(1,1-dimethylethyl)-N-(1- |
| | butyl-2,6-dinitroaniline | methylpropyl)-2,6- |
| | | dinitrobenzenamine |
| Chloralin | | |
| dinitramine | N1,N1-diethyl-2,6-dinitro-4- | N3,N3-diethyl-2,4-dinitro-6- |
| | trifluoromethyl-m- | (trifluoromethyl)-1,3- |
| | phenylenediamine | benzenediamine |
| ethalfluralin (Sonalan) | N-ethyl-α,α,α-trifluoro-N-(2- | N-ethyl-N-(2-methyl-2- |
| | methylallyl)-2,6-dinitro-p- | propenyl)-2,6-dinitro-4- |
| | toluidine | (trifluoromethyl)benzenamine |
| fluchloralin | N-(2-chloroethyl)-2,6-dinitro-N- | N-(2-chloroethyl)-2,6-dinitro-N- |
| | propyl-4-(trifluoromethyl)aniline | propyl-4- |
| | or | (trifluoromethyl)benzenamine |
| | N-(2-chloroethyl)-α,α,α- | |
| | trifluoro-2,6-dinitro-N-propyl-p- | |
| | toluidine | |
| isopropalin | 4-isopropyl-2,6-dinitro-N,N- | 4-(1-methylethyl)-2,6-dinitro-N,N- |
| | dipropylaniline | dipropylbenzenamine |
| methalpropalin | α,α,α-trifluoro-N-(2- | N-(2-methyl-2-propenyl)-2,6-dinitro- |
| | methylallyl)-2,6-dinitro-N- | N-propyl-4- |
| | propyl-p-toluidine | (trifluoromethyl)benzenamine |
| nitralin | 4-methylsulfonyl-2,6-dinitro- | 4-(methylsulfonyl)-2,6-dinitro-N,N- |
| | N,N-dipropylaniline | dipropylbenzenamine |
| oryzalin (SURFLAN) | 3,5-dinitro-N4,N4- | 4-(dipropylamino)-3,5- |
| | dipropylsulfanilamide | dinitrobenzenesulfonamide |
| pendimethalin (PROWL) | N-(1-ethylpropyl)-2,6- | N-(1-ethylpropyl)-3,4-dimethyl-2,6- |
| | dinitro-3,4-xylidine | dinitrobenzenamine |
| prodiamine | 5-dipropylamino-α,α,α- | 2,4-dinitro-N3,N3-dipropyl-6- |
| | trifluoro-4,6-dinitro-o-toluidine | (trifluoromethyl)-1,3- |
| | or | benzenediamine |
| | 2,6-dinitro-N1,N1-dipropyl-4- | |
| | trifluoromethyl-m- | |
| | phenylenediamine | |
| profluralin | N-cyclopropylmethyl-α,α,α- | N-(cyclopropylmethyl)-2,6-dinitro-N- |
| | trifluoro-2,6-dinitro-N-propyl-p- | propyl-4- |
| | toluidine | (trifluoromethyl)benzenamine |
| | or | |
| | N-cyclopropylmethyl-2,6-dinitro- | |
| | N-propyl-4-trifluoromethylaniline | |
| trifluralin (TREFLAN, | α,α,α-trifluoro-2,6-dinitro-N,N- | 2,6-dinitro-N,N-dipropyl-4- |
| TRIFIC, TRILLIN) | dipropyl-p-toluidine | (trifluoromethyl)benzenamine |

Phosphoroamidates

| | | |
|---|---|---|
| AMP (Amiprofos methyl); | | |
| amiprophos-methyl | | |
| Butamifos | O-ethyl O-6-nitro-m-tolyl (RS)- | O-ethyl O-(5-methyl-2-nitrophenyl) |
| | sec-butylphosphoramidothioate | (1-methylpropyl)phosphoramidothioate |

Pyridines

| | | |
|---|---|---|
| Dithiopyr | | |
| Thiazopyr | methyl 2-difluoromethyl-5-(4,5- | methyl 2-(difluoromethyl)-5-(4,5- |
| | dihydro-1,3-thiazol-2-yl)-4- | dihydro-2-thiazolyl)-4-(2- |
| | isobutyl-6- | methylpropyl)-6-(trifluoromethyl)-3- |
| | trifluoromethylnicotinate | pyridinecarboxylate |

The chromosome doubling agent may come in contact with the embryo at various times. If the embryo is isolated the doubling agent may come in contact immediately after isolation and before germination. If the embryo is contained within the seed, it may come in contact with the doubling agent any time after pollination and before dry-down. The embryo, whether it is isolated or not, may come in contact with the doubling agent any time between 6 hours after pollination and 21 days after pollination. The duration of contact between the chromosomal doubling agent may vary. Contact may be from less than 24 hours, for example 4-12 hours, to about a week. The duration of contact is generally from about 24 hours to 2 days.

Methods for obtaining homozygous plants, plant cells, and seeds are provided herein.

One method comprises obtaining a sorghum doubled haploid embryo, seed, or plant by contacting a sorghum haploid embryo, produced by pollinating stigmas of a sorghum female diploid plant with 2PWCS25R, 2PNUM88R, or 2PCNW79R, with a chromosome doubling agent and obtaining a doubled haploid embryo, seed, or plant.

In another method, sorghum doubled haploid plants are obtained using a method comprising the following steps: a) pollinating stigmas of a sorghum diploid plant with pollen from inducer line 2PWCS25R, 2PNUM88R, or 2PCNW79R, wherein the inducer line has a marker gene that is expressed in embryos and/or endosperm tissue; b) selecting a sorghum haploid embryo/seeds which does not express a marker gene; c) contacting the sorghum haploid embryo with a gas, solution or solid comprising a chromosome doubling agent; and d) regenerating the resulting sorghum doubled haploid embryo into a doubled haploid plant.

In another method, sorghum doubled haploid seed is obtained using a method comprising the following steps: a) obtaining a sorghum haploid seed by pollinating an ovule with inducer line 2PWCS25R, 2PNUM88R, or 2PCNW79R wherein the ovule comprises a set of maternal chromosomes and wherein the inducer line comprises a set of paternal chromosomes; b) contacting the sorghum haploid seed with a medium comprising a chromosome doubling agent; c) selecting a sorghum doubled haploid seed wherein the sorghum doubled haploid seed comprises a triploid endosperm and a doubled haploid embryo. The sorghum doubled haploid seed produced by such a method wherein the triploid endosperm comprises two sets of maternal chromosomes and one set of paternal chromosomes, and wherein the sorghum doubled haploid embryo has a first and second set of maternal chromosomes and wherein the first set of maternal chromosomes is homozygous to the second set of maternal chromosomes can be produced. Also included is a method of determining the origin of the chromosomes with the use of a marker that is expressed during early seed development.

In another method, a population of doubled haploid sorghum plants is obtained using a method comprising the following steps: a) obtaining a set of sorghum haploid kernels by pollinating stigmas of a panicle with inducer line 2PWCS25R, 2PNUM88R, or 2PCNW79R, wherein the panicle comprises a set of maternal chromosomes and wherein the inducer line comprises a set of paternal chromosomes; b) contacting said set of sorghum haploid kernels with a medium comprising a chromosome doubling agent; c) selecting a set of sorghum doubled haploid kernels wherein each kernel of said set of sorghum doubled haploid kernels comprises a triploid endosperm and a doubled haploid embryo; d) growing said set of sorghum doubled haploid kernels into a population of doubled haploid sorghum plants. The set of sorghum doubled haploid kernels produced have triploid endosperm and the triploid endosperm comprises two sets of maternal chromosomes and one set of paternal chromosomes. The doubled haploid embryo also has two sets of maternal chromosomes. These two sets of chromosomes are homozygous. The first set of chromosomes being replicated to form the second set of chromosomes. The various sets obtained from these methods can include the set of embryos on a sorghum panicle, the set of kernels on a sorghum panicle, and the set of sorghum doubled haploids plants.

In another method, a doubled haploid sorghum plant is obtained using a method comprising: a) pollinating stigmas of a sorghum panicle with inducer line 2PWCS25R, 2PNUM88R, or 2PCNW79R; b) contacting the sorghum panicle with a medium comprising a chromosome doubling agent; c) generating an embryo from the sorghum panicle into a doubled haploid sorghum plant. Other aspects of the method include removing the sorghum panicle from the plant with or without the stalk or some portion of the stalk attached. The panicle can be removed before, during, or after pollination and placed into a solution. The panicle can be placed in a solution 6 hours to 21 days after pollination and up to 35 days after pollination. The solution may comprise water or water and nutrients. The solution may come into contact with the panicle directly or indirectly, for example via filter paper or cotton. The chromosome doubling agent can come in contact with the panicle after pollination and before or after the panicle is removed from the plant. The chromosomal doubling agent may come into contact with the panicle directly or indirectly, for example via filter paper or cotton.

In another method, a set of doubled haploid sorghum embryos is obtained using a method comprising the following steps: a) obtaining a set of sorghum haploid embryos by pollinating a panicle with pollen from inducer line 2PWCS25R, 2PNUM88R, or 2PCNW79R; wherein the panicle comprises a set of maternal chromosomes from an F1 sorghum plant; and wherein the inducer line comprises a set of paternal chromosomes; b) contacting said set of sorghum haploid embryos with a medium comprising a chromosome doubling agent; c) selecting a set of doubled haploid sorghum embryos wherein each doubled haploid sorghum embryo of said set of doubled haploid sorghum embryos is genetically different from each of the other doubled haploid sorghum embryos of said set of doubled haploid sorghum embryos; d) growing said set of doubled haploid sorghum embryos into a population of doubled haploid sorghum plants. This method develops a unique set of doubled haploid sorghum embryos. This unique set of embryos is derived directly from one sorghum panicle, wherein “being derived directly” indicates that a filial generation does not occur between development of the haploid embryos and the development of the set of doubled haploid embryos.

A method of inbred selection is also provided in which the method comprises the following steps: a) cross pollinating two inbred sorghum plants; b) growing the F1 seed; c) pollinating the F1 plant with inducer line 2PWCS25R, 2PNUM88R, or 2PCNW79R to produce sorghum haploid embryos; d) contacting the sorghum haploid embryos with a chromosome doubling agent to produce sorghum doubled haploid embryos; e) generating sorghum doubled haploid plants; f) evaluating said sorghum doubled haploid plants for agronomic performance and combining ability. The development of haploids step may also be done at later generations, F2, F3, F4, etc. Producing haploids from later generations allows for additional opportunities for recombination.

### Breeding With 2PWCS25R, 2PNUM88R, or 2PCNW79R

Field crops are bred through techniques that take advantage of the plant's method of pollination. A plant is self-pollinating if pollen from one flower is transferred to the same or another flower of the same plant. A plant is cross-pollinated if the pollen comes from a flower on a different plant.

Plants that have been self-pollinated and selected for type for many generations become homozygous at almost all gene loci and produce a uniform population of true breeding progeny. A cross between two homozygous plants from differing backgrounds or two homozygous lines produces a uniform population of hybrid plants that may be heterozygous for many gene loci. A cross of two plants that are each heterozygous at a number of gene loci will produce a population of hybrid plants that differ genetically and will not be uniform.

Grain sorghum (Sorghum bicolor) is a chiefly self-pollinating species. Sorghum plants are bred in most cases by self-pollination techniques. With the incorporation of male sterility (either genetic or cytoplasmic) cross pollination breeding techniques can also be utilized. Sorghum has a perfect flower with both male and female parts in the same flower located in the panicle. The flowers are usually in pairs on the panicle branches. A floret is a triplet-2 flowers that abort, with a third flower in the middle that produces 1 kernel. Natural pollination occurs in sorghum when anthers open and pollen falls onto receptive stigma. Because of the close proximity of anthers and stigma in the panicle, self-pollination is very high (average 94%). Cross pollination may occur when wind or convection currents move pollen from the anthers of one plant to receptive stigma on another plant. Cross pollination is greatly enhanced with incorporation of male sterility which renders anthers nonviable without affecting the receptivity of the stigma. Successful pollination in the case of sterile anthers uses cross pollination.

Sorghum is in the same family as maize and has a similar growth habit, but with more tillers and a more extensively branched root system. Sorghum is more drought resistant and heat-tolerant than maize. It requires an average temperature of at least 25° C. to produce maximum yields. Sorghum's ability to thrive with less water than maize may be due to its ability to hold water in its foliage better than maize. Sorghum has a waxy coating on its leaves and stems which helps to keep water in the plant even in intense heat. Wild species of sorghum tend to grow to a height of approximately 2 meters; however in order to improve harvestability, dwarfing genes have been selected in cultivated varieties and hybrids such that most cultivated varieties and hybrids grow to between 60 and 150 cm tall.

### Inbred Development

The development of sorghum hybrids requires the development of homozygous inbred lines, the crossing of these lines, and the evaluation of the crosses. Pedigree breeding methods, and to a lesser extent population breeding methods, are used to develop inbred lines from breeding populations. Breeding programs combine desirable traits from two or more inbred lines into breeding pools from which new inbred lines are developed by selfing and selection of desired phenotypes. The new inbreds become potential parents of commercial hybrids. That is, the new inbreds can be crossed with other inbred lines and the hybrids from these crosses can be evaluated to determine which have commercial potential.

Pedigree breeding starts with the crossing of two genotypes (e.g., inbred lines), each of which may have one or more desirable characteristics that is lacking in the other or which complement the other. If the two original parents do not provide all of the desired characteristics, other sources can be included in the breeding population. In the pedigree method, superior plants are self-crossed and selected in successive generations. In the succeeding generations the heterozygous condition gives way to homogeneous lines as a result of self-pollination and selection. Typically, in the pedigree method of breeding five or more generations of selfing and selection is practiced. F₁ to F₂; F₂ to F₃; F₃ to F₄, F₄ to F₅, etc.

Backcrossing can be used to improve an inbred line. Backcrossing transfers a specific desirable trait from one inbred (e.g., a donor source) to an inbred that lacks that trait (e.g., a recurrent parent). This can be accomplished for example by first crossing a superior inbred (A) (recurrent parent) to a donor inbred (non-recurrent parent), which carries the appropriate genes(s) for the trait in question. The progeny of this cross is then mated back to the superior recurrent parent (A) followed by selection in the resultant progeny for the desired trait to be transferred from the non-recurrent parent. After three or more backcross generations with selection for the desired trait, the progeny will be heterozygous for loci controlling the characteristic being transferred but will be like the superior parent for most or almost all other genes. The last backcross generation can be self-crossed and selected for the trait to give pure breeding progeny for the trait being transferred.

### Controlling Self-Pollination

Sorghum varieties are mainly self-pollinated; therefore, self-pollination of the parental varieties must be controlled to make hybrid development feasible. A pollination control system and effective transfer of pollen from one parent to the other offers improved plant breeding and an effective method for producing hybrid seed and plants. For example, the milo or A₁ cytoplasmic male sterility (CMS) system, developed via a cross between milo and kafir cultivars, is one of the most frequently used CMS systems in hybrid sorghum production (Stephens J C & Holland P F, Cytoplasmic Male Sterility for Hybrid Sorghum Seed Production, Agron. J. 46:20-23 (1954)). Other CMS systems for sorghum include, but are not limited to, A₂, isolated from IS 12662c (Schertz K F, Registration of A₂Tₓ2753 and BTₓ2753 Sorghum Germplasm, Crop Sci. 17:983 (1977)), A₃, isolated from IS 1112c or converted Nilwa (Quinby J R, Interactions of Genes and Cytoplasms in Male-Sterility in Sorghums, Proc. 35th Corn Sorghum Res. Conf. Am. Seed Trade Assoc. Chicago, III., pp. 5-8 (1980)), A₄, isolated from IS 7920c (Worstell et al, Relationship among Male-Sterility Inducing Cytoplasms of Sorghum, Crop Sci. 24:186-189 (1984)).

In developing improved new sorghum hybrid varieties, breeders may use a CMS plant as the female parent. In using these plants, breeders attempt to improve the efficiency of seed production and the quality of the F₁ hybrids and to reduce the breeding costs. If one of the parents is a CMS plant that is incapable of producing pollen, only cross pollination will occur. By eliminating the pollen of one parental variety in a cross, a plant breeder is assured of obtaining hybrid seed of uniform quality, provided that the parents are of uniform quality and the breeder conducts a single cross.

In one instance, production of F₁ hybrids includes crossing a CMS female parent with a pollen-producing male parent. To reproduce effectively, however, the male parent of the F₁ hybrid must have a fertility restorer gene (Rf gene). The presence of an Rf gene means that the F₁ generation will not be completely or partially sterile, so that either self-pollination or cross pollination may occur. Self-pollination of the F₁ generation to produce several subsequent generations is important to ensure that a desired trait is heritable and stable and that a new variety has been isolated.

Promising advanced breeding lines commonly are tested and compared to

appropriate standards in environments representative of the commercial target area(s). The best lines are candidates for new commercial lines; and those still deficient in a few traits may be used as parents to produce new populations for further selection.

### Hybrid Development

A hybrid sorghum variety is the cross of two inbred lines, each of which may have one or more desirable characteristics lacked by the other or which complement the other. The hybrid progeny of the first generation is designated F₁. In the development of hybrids only the F₁ hybrid plants are sought. The F₁ hybrid is more vigorous than its inbred parents. This hybrid vigor, or heterosis, can be manifested in many ways, including increased vegetative growth and increased yield.

The development of a hybrid sorghum variety involves five steps: (1) the formation of “restorer” and “non-restorer” germplasm pools; (2) the selection of superior plants from various “restorer” and “non-restorer” germplasm pools; (3) the selfing of the superior plants for several generations to produce a series of inbred lines, which although different from each other, each breed true and are highly uniform; (4) the conversion of inbred lines classified as non-restorers to cytoplasmic male sterile (CMS) forms, and (5) crossing the selected cytoplasmic male sterile (CMS) inbred lines with selected fertile inbred lines (restorer lines) to produce the hybrid progeny (F₁).

Because sorghum is normally a self-pollinated plant and because both male and female flowers are in the same panicle, large numbers of hybrid seed can only be produced by using cytoplasmic male sterile (CMS) inbreds. Flowers of the CMS inbred are fertilized with pollen from a male fertile inbred carrying genes which restore male fertility in the hybrid (F₁) plants. An important consequence of the homozygosity and homogeneity of the inbred lines is that the hybrid between any two inbreds will always be the same. Once the inbreds that produce the best hybrid have been identified, the hybrid seed can be reproduced indefinitely as long as the homogeneity of the inbred parent is maintained.

A single cross hybrid is produced when two inbred lines are crossed to produce the F₁ progeny. Much of the hybrid vigor exhibited by F₁ hybrids is lost in the next generation (F₂). Consequently, seed from hybrid varieties is not used for planting stock.

Hybrid grain sorghum can be produced using wind to move the pollen. Alternating rows of the cytoplasmic male sterile inbred (female) and the male fertile inbred (male) are planted in the same field. Wind moves the pollen shed by the male inbred to receptive stigma on the female. Providing that there is sufficient isolation from sources of foreign sorghum pollen, the stigma of the male sterile inbred (female) will be fertilized only with pollen from the male fertile inbred (male). The resulting seed, born on the male sterile (female) plants is therefore hybrid and will form hybrid plants that have full fertility restored.

### Locus Conversions of Sorghum Line 2PWCS25R, 2PNUM88R, and 2PCNW79R

2PWCS25R, 2PNUM88R, and 2PCNW79R represent new base genetic lines into which a new locus or trait may be introduced. Direct transformation and backcrossing represent two important methods that can be used to accomplish such an introgression. The term locus conversion is used to designate the product of such an introgression.

To select and develop a superior hybrid, it is necessary to identify and select genetically unique individuals that occur in a segregating population. The segregating population is the result of a combination of crossover events plus the independent assortment of specific combinations of alleles at many gene loci that results in specific and unique genotypes. Once such a variety is developed its value to society is substantial since it is important to advance the germplasm base as a whole in order to maintain or improve traits such as yield, disease resistance, pest resistance and plant performance in extreme weather conditions. Locus conversions are routinely used to add or modify one or a few traits of such a line and this further enhances its value and usefulness to society.

Backcrossing can be used to improve inbred varieties and a hybrid variety which is made using those inbreds. Backcrossing can be used to transfer a specific desirable trait from one variety, the donor parent, to an inbred called the recurrent parent which has overall good agronomic characteristics yet that lacks the desirable trait. This transfer of the desirable trait into an inbred with overall good agronomic characteristics can be accomplished by first crossing a recurrent parent to a donor parent (non-recurrent parent). The progeny of this cross is then mated back to the recurrent parent followed by selection in the resultant progeny for the desired trait to be transferred from the non-recurrent parent.

Traits may be used by those of ordinary skill in the art to characterize progeny. Traits are commonly evaluated at a significance level, such as a 1%, 5% or 10% significance level, when measured in plants grown in the same environmental conditions. For example, a locus conversion of 2PWCS25R, 2PNUM88R, or 2PCNW79R may be characterized as having essentially the same phenotypic traits as 2PWCS25R, 2PNUM88R, or 2PCNW79R, respectively. Molecular markers can also be used during the breeding process for the selection of qualitative traits. For example, markers can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants.

A locus conversion of 2PWCS25R, 2PNUM88R, or 2PCNW79R will retain the genetic integrity of 2PWCS25R, 2PNUM88R, or 2PCNW79R, respectively. For example, a locus conversion of 2PWCS25R, 2PNUM88R, or 2PCNW79R can be developed when DNA sequences are introduced through backcrossing (Hallauer et al., 1988), with a parent of 2PWCS25R, 2PNUM88R, or 2PCNW79R utilized as the recurrent parent. Both naturally occurring and transgenic DNA sequences may be introduced through backcrossing techniques. A backcross conversion may produce a plant with a locus conversion in at least one or more backcrosses, including at least 2 crosses, at least 3 crosses, at least 4 crosses, at least 5 crosses and the like. Molecular marker assisted breeding or selection may be utilized to reduce the number of backcrosses necessary to achieve the backcross conversion. For example, see Openshaw, S. J. et al., Marker-assisted Selection in Backcross Breeding. In: Proceedings Symposium of the Analysis of Molecular Data, August 1994, Crop Science Society of America, Corvallis, OR, where it is demonstrated that a backcross conversion can be made in as few as two backcrosses. A locus conversion of 2PWCS25R, 2PNUM88R, or 2PCNW79R can be determined through the use of a molecular profile. Examples of molecular markers that could be used to determine the molecular profile include Restriction Fragment Length Polymorphisms (RFLP), Polymerase Chain Reaction (PCR) analysis, and Simple Sequence Repeats (SSR), and Single Nucleotide Polymorphisms (SNPs).

### Genetic Modification of Sorghum Line 2PWCS25R, 2PNUM88R, or 2PCNW79R

Genetic modification, such as genome editing and transformation methods facilitate engineering of the genome of plants to contain and express modified genetic elements, such as transgenes, foreign genetic elements, or additional copies of endogenous elements, or modified versions of native or endogenous genetic elements in order to alter at least one trait of a plant in a specific manner. Any sequences, such as DNA, whether from a different species or from the same species, which have been stably inserted into a genome using transformation are referred to herein collectively as “transgenes” and/or “transgenic events”. Transgenes can be moved from one genome to another using breeding techniques which may include crossing, backcrossing or double haploid production. In some embodiments, a transformed variant of 2PWCS25R, 2PNUM88R, or 2PCNW79R may comprise at least one transgene or genetic modification but could contain at least 1, 2, 3, 4, 5, 6, 7, 8, 9, 10 transgenes or genetic modifications and no more than 15, 14, 13, 12, 11, 10, 9, 8, 7, 6, 5, 4, 3, or 2 transgenes or genetic modifications. Transformed versions of the claimed variety 2PWCS25R, 2PNUM88R, or 2PCNW79R as well as hybrid combinations containing and inheriting the transgene thereof are provided. F1 hybrid seed are provided which are produced by crossing a different plant with variety 2PWCS25R, 2PNUM88R, or 2PCNW79R comprising a transgene introduced into variety 2PWCS25R, 2PNUM88R, or 2PCNW79R by backcrossing or genetic transformation and is inherited by the F₁ hybrid seed.

In general, methods to transform, modify, edit or alter plant endogenous genomic DNA include altering the plant native DNA sequence or a pre-existing transgenic sequence including regulatory elements, coding and non-coding sequences. These methods can be used, for example, to target nucleic acids to pre-engineered target recognition sequences in the genome. Such pre-engineered target sequences may be introduced by genome editing or modification. Genome editing or genome editing techniques involve the manipulation of the genetic material of a plant, plant part, plant seed or plant cell by deleting, replacing, or inserting a DNA sequence or base in the genome of the plant, plant part, plant seed or plant cell. As an example, a genetically modified or edited plant variety can be generated using “custom” or engineered endonucleases such as meganucleases produced to modify plant genomes (see e.g., WO 2009/114321; Gao et al. (2010) Plant Journal 1:176-187). Another site-directed genome engineering method is through the use of zinc finger domain recognition coupled with the restriction properties of restriction enzyme. See e.g., Urnov, et al., (2010) Nat Rev Genet. 11(9): 636-46; Shukla, et al., (2009) Nature 459 (7245): 437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39 (12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9:39; The Cas9/guide RNA-based system allows targeted cleavage of genomic DNA guided by a customizable small noncoding RNA in plants (see e.g., WO 2015026883A1). Provided are methods for modifying seeds, plants, plant parts, seed cells or plant cells, such as those grown from the seed disclosed herein, in which genome editing techniques are performed on the seed, plant, plant part or cells thereby modifying the seed, plant, plant part or cells. Methods for modifying the genome of seeds, plants, plant parts, seed cells or the genome of plant cells grown from the seed disclosed herein include performing genome editing techniques on the genome of such materials, such that the genome is modified. The seed, plant, plant part, plant cell or seed cells can be contacted with components sufficient to effect editing of the genome. The components can include an enzyme capable of effecting a DNA break, such as a double-stranded DNA break, in the nuclear genetic material. Modified plants, plant parts and seeds can be grown from the gene edited materials.

Plant transformation methods may involve the construction of an expression vector. Such a vector comprises a DNA sequence that contains a gene under the control of or operatively linked to a regulatory element, for example a promoter. The vector may contain one or more genes and one or more regulatory elements.

A genetic trait (e.g., a transgenic event) which has been engineered into a particular sorghum plant using transformation techniques, could be moved into another line using traditional breeding techniques that are well known in the plant breeding arts. For example, a backcrossing approach could be used to move a transgene from a transformed sorghum plant to an elite inbred line and the resulting progeny would comprise a transgene. Also, if an inbred line was used for the transformation then the transgenic plants could be crossed to a different line in order to produce a transgenic hybrid sorghum plant. As used herein, “crossing” can refer to a simple X by Y cross, or the process of backcrossing, depending on the context. Various genetic elements can be introduced into the plant genome using transformation. These elements include but are not limited to genes; coding sequences; inducible, constitutive, and tissue specific promoters; enhancing sequences; and signal and targeting sequences. For example, see, U.S. Pat. No. 6,118,055.

With transgenic plants according to the present discovery, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods.

A genetic map can be generated, primarily via conventional Restriction Fragment Length Polymorphisms (RFLP), Polymerase Chain Reaction (PCR) analysis, and Simple Sequence Repeats (SSR), and Single Nucleotide Polymorphisms (SNPs), which identifies the approximate chromosomal location of the integrated DNA molecule coding for the foreign protein. Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR, SNP, and sequencing, all of which are conventional techniques.

Likewise, by means of the present discovery, plants can be genetically engineered to express various phenotypes of agronomic interest. Exemplary transgenes implicated in this regard include, but are not limited to, those categorized below.
1. Genes that create a site for site specific DNA integration.

This includes the introduction of FRT sites that may be used in the FLP/FRT system and/or Lox sites that may be used in the Cre/Loxp system. For example, see, Lyznik, et al., (2003) “Site-Specific Recombination for Genetic Engineering in Plants”, Plant Cell Rep 21:925-932 and WO 99/25821, which are hereby incorporated by reference. Other systems that may be used include the Gin recombinase of phage Mu (Maeser, et al., 1991), the Pin recombinase of E. coli (Enomoto, et al., 1983), and the R/RS system of the pSR1 plasmid (Araki, et al., 1992).
2. Genes that affect abiotic stress resistance (including but not limited to flowering, panicle/glume and seed development, enhancement of nitrogen utilization efficiency, altered nitrogen responsiveness, drought resistance or tolerance, cold resistance or tolerance, and salt resistance or tolerance) and increased yield under stress.

For example, see, WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO2000060089, WO2001026459, WO2001035725, WO2001034726, WO2001035727, WO2001036444, WO2001036597, WO2001036598, WO2002015675, WO2002017430, WO2002077185, WO2002079403, WO2003013227, WO2003013228, WO2003014327, WO2004031349, WO2004076638, WO9809521 and WO9938977 describing genes, including CBF genes and transcription factors effective in mitigating the negative effects of freezing, high salinity, and drought on plants, as well as conferring other positive effects on plant phenotype; US Patent Application Publication No. 2004/0148654 and WO01/36596 where abscisic acid is altered in plants resulting in improved plant phenotype such as increased yield and/or increased tolerance to abiotic stress; WO2000/006341, WO04/090143, U.S. patents application Ser. Nos. 10/817,483 and 09/545,334 where cytokinin expression is modified resulting in plants with increased stress tolerance, such as drought tolerance, and/or increased yield. Also see WO0202776, WO03052063, JP2002281975, U.S. Pat. No. 6,084,153, WO0164898, U.S. Pat. Nos. 6,177,275 and 6,107,547 (enhancement of nitrogen utilization and altered nitrogen responsiveness). For ethylene alteration, see, US Patent Application Publication Numbers 2004/0128719, 2003/0166197 and WO200032761. For plant transcription factors or transcriptional regulators of abiotic stress, see e.g., US Patent Application Publication Number 2004/0098764 or US Patent Application Publication Number 2004/0078852.

Other genes and transcription factors that affect plant growth and agronomic traits such as yield, flowering, plant growth and/or plant structure, can be introduced or introgressed into plants, see, e.g., WO97/49811 (LHY), WO98/56918 (ESD4), WO97/10339 and U.S. Pat. No. 6,573,430 (TFL), U.S. Pat. No. 6,713,663 (FT), WO96/14414 (CON), WO96/38560, WO01/21822 (VRN1), WO00/44918 (VRN2), WO99/49064 (GI), WO00/46358 (FRI), WO97/29123, U.S. Pat. Nos. 6,794,560, 6,307,126 (GAI), WO99/09174 (D8 and Rht), and WO2004076638 and WO2004031349 (transcription factors).
3. Transgenes that confer or contribute to an altered grain characteristic, such as:
A. Altered phosphorus content, for example, by the
(1) Introduction of a phytase-encoding gene would enhance breakdown of phytate, adding more free phosphate to the transformed plant. For example, see, Van Hartingsveldt, et al., Gene 127:87 (1993), for a disclosure of the nucleotide sequence of an Aspergillus niger phytase gene.
(2) Up-regulation of a gene that reduces phytate content. In maize, this, for example, could be accomplished, by cloning and then re-introducing DNA associated with one or more of the alleles, such as the LPA alleles, identified in maize mutants characterized by low levels of phytic acid, such as in Raboy, et al. (1990).
B. Altered fatty acids, for example, by down-regulation of stearoyl-ACP desaturase to increase stearic acid content of the plant. See Knultzon, et al., Proc. Natl. Acad. Sci. USA 89:2624 (1992).
C. Altered carbohydrates effected, for example, by altering a gene for an enzyme that affects the branching pattern of starch, a gene altering thioredoxin. (See, U.S. Pat. No. 6,531,648). See, Shiroza, et al., (1988) J. Bacteriol 170:810 (nucleotide sequence of Streptococcus mutans fructosyltransferase gene), Steinmetz, et al., (1985) Mol. Gen. Genet. 200:220 (nucleotide sequence of Bacillus subtilis levansucrase gene), Pen, et al., (1992) Bio/Technology 10:292 (production of transgenic plants that express Bacillus licheniformis alpha-amylase), Elliot, et al., (1993) Plant Molec Biol 21:515 (nucleotide sequences of tomato invertase genes), Søgaard, et al., (1993) J. Biol. Chem. 268:22480 (site-directed mutagenesis of barley alpha-amylase gene) and Fisher, et al., (1993) Plant Physiol 102:1045 (maize endosperm starch branching enzyme II), WO 99/10498 (improved digestibility and/or starch extraction through modification of UDP-D-xylose 4-epimerase, Fragile 1 and 2, Ref1, HCHL, C4H), U.S. Pat. No. 6,232,529 (method of producing high oil seed by modification of starch levels (AGP)). The fatty acid modification genes mentioned above may also be used to affect starch content and/or composition through the interrelationship of the starch and oil pathways.
D. Altered antioxidant content or composition, such as alteration of tocopherol or tocotrienols. For example, see, U.S. Pat. No. 6,787,683, US Patent Application Publication Number 2004/0034886 and WO 00/68393 involving the manipulation of antioxidant levels through alteration of a phytl prenyl transferase (ppt), WO 03/082899 through alteration of a homogentisate geranyl geranyl transferase (hggt).
E. Altered essential seed amino acids. For example, see, U.S. Pat. No. 6,127,600 (method of increasing accumulation of essential amino acids in seeds), U.S. Pat. No. 6,080,913 (binary methods of increasing accumulation of essential amino acids in seeds), U.S. Pat. No. 5,990,389 (high lysine), WO99/40209 (alteration of amino acid compositions in seeds), WO99/29882 (methods for altering amino acid content of proteins), U.S. Pat. No. 5,850,016 (alteration of amino acid compositions in seeds), WO98/20133 (proteins with enhanced levels of essential amino acids), U.S. Pat. No. 5,885,802 (high methionine), U.S. Pat. No. 5,885,801 (high threonine), U.S. Pat. No. 6,664,445 (plant amino acid biosynthetic enzymes), U.S. Pat. No. 6,459,019 (increased lysine and threonine), U.S. Pat. No. 6,441,274 (plant tryptophan synthase beta subunit), U.S. Pat. No. 6,346,403 (methionine metabolic enzymes), U.S. Pat. No. 5,939,599 (high sulfur), U.S. Pat. No. 5,912,414 (increased methionine), WO98/56935 (plant amino acid biosynthetic enzymes), WO98/45458 (engineered seed protein having higher percentage of essential amino acids), WO98/42831 (increased lysine), U.S. Pat. No. 5,633,436 (increasing sulfur amino acid content), U.S. Pat. No. 5,559,223 (synthetic storage proteins with defined structure containing programmable levels of essential amino acids for improvement of the nutritional value of plants), WO96/01905 (increased threonine), WO95/15392 (increased lysine), US Patent Application Publication Number 2003/0163838, US Patent Application Publication Number 2003/0150014, US Patent Application Publication Number 2004/0068767, U.S. Pat. No. 6,803,498, WO01/79516, and WO00/09706 (Ces A: cellulose synthase), U.S. Pat. No. 6,194,638 (hemicellulose), U.S. Pat. No. 6,399,859 and US Patent Application Publication Number 2004/0025203 (UDPGdH), U.S. Pat. No. 6,194,638 (RGP).
4. Genes that confer male sterility

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describes a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on,” the promoter, which in turn allows the gene that confers male fertility to be transcribed.

A. A dominant nuclear gene, Ms(tc) controlling male sterility. See, Elkonin, L. A., Theor. Appl. Genet. (2005) 111(7): 1377-1384.
B. A tapetum-specific gene, RTS, a sorghum anther-specific gene is required for male fertility and its promoter sequence directs tissue-specific gene expression in different plant species. Luo, Hong, et al., Plant Molecular Biology., 62(3): 397-408(12) (2006). Introduction of a deacetylase gene under the control of a tapetum-specific promoter and with the application of the chemical N-Ac-PPT. See International Publication No. WO 01/29237.
C. Introduction of various stamen-specific promoters. Anther-specific promoters which are of particular utility in the production of transgenic male-sterile monocots and plants for restoring their fertility. See, U.S. Pat. No. 5,639,948. See also, International Publication Nos. WO 92/13956 and WO 92/13957.
D. Introduction of the barnase and the barstar genes. See, Paul, et al., Plant Mol. Biol., 19:611-622 (1992).

For additional examples of nuclear male and female sterility systems and genes, see also, U.S. Pat. Nos. 5,859,341, 6,297,426, 5,478,369, 5,824,524, 5,850,014, and 6,265,640. See also, Hanson, Maureen R., et al., “Interactions of Mitochondrial and Nuclear Genes That Affect Male Gametophyte Development,” Plant Cell., 16:S154-S169 (2004), all of which are hereby incorporated by reference.
A. Modification of RNA editing within mitochondrial open reading frames. See, Pring, D. R., et al, Curr. Genet. (1998) 33 (6): 429-436; Pring, D. R., et al., J. Hered. (1999) 90 (3): 386-393; Pring, D. R., et al., Curr. Genet. (2001) 39(5-6): 371-376; and Hedgcoth, C., et al., Curr. Genet. (2002) 41(5): 357-365.

B. Cytoplasmic male sterility (CMS) from mutations at atp6 codons. See, Kempken, F., FEBS. Lett. (1998): 441(2): 159-160.
C. Inducing male sterility through heat shock. See, Wang, L., Yi Chuan Xue Bao. (2000) 27(9): 834-838.
D. Inducing male sterility through treatment of streptomycin on sorghum callus cultures. See, Elkonin, L. A., et al., Genetica (2008) 44(5): 663-673.

### Uses of Sorghum

Sorghum is used as livestock feed, as sugar or grain for human consumption, as biomass, and as raw material in industry. Sorghum grain can be used as livestock feed, such as to beef cattle, dairy cattle, hogs and poultry. In some embodiments, the plant is used as livestock feed in the form of fodder, silage, hay and pasture. In some embodiments, commodity plant products produced from hybrid seed such as food, feed, forage, and syrup are provided.

Provided are uses of sorghum in the form of bread, porridge, confectionaries and as an alcoholic beverage. Grain sorghum may be ground into flour and either used directly or blended with wheat or corn flour in the preparation of food products. In addition to direct consumption of the grain, sorghum has long been used in many areas of the world to make beer. The uses of sorghum , in addition to human consumption of kernels, include both products of dry and wet milling industries. The principal products of sorghum dry milling are grits, meal and flour. Starch and other extracts for food use can be provided by the wet milling process.

Also provided are uses of sorghum as an industrial raw material. Industrial uses include sorghum starch from the wet-milling industry and sorghum flour from the dry milling industry. Sorghum starch and flour have application in the paper and textile industries. Other industrial uses include applications in adhesives, building materials and as oil-well muds. Considerable amounts of sorghum , both grain and plant material, have been used in industrial alcohol production.

Provided are seed of sorghum lines 2PWCS25R, 2PNUM88R, and 2PCNW79R; plants of sorghum lines 2PWCS25R, 2PNUM88R, and 2PCNW79R; plant parts of sorghum lines 2PWCS25R, 2PNUM88R, or 2PCNW79R; and processes for making a plant that comprise crossing sorghum line 2PWCS25R, 2PNUM88R, or 2PCNW79R with another plant. In some embodiments, 2PWCS25R, 2PNUM88R, or 2PCNW79R may be provided with cytoplasm comprising a gene or genes that cause male sterility. Also disclosed are processes for making a plant containing in its genetic material one or more traits introgressed into 2PWCS25R, 2PNUM88R, or 2PCNW79R through backcross conversion and/or transformation, and to the seed, plant and plant arts produced thereby. Hybrid sorghum seed, plant, or plant part produced by crossing line 2PWCS25R, 2PNUM88R, or 2PCNW79R or a locus conversion of 2PWCS25R, 2PNUM88R, or 2PCNW79R with another plant are also provided.

### TFMSA Emasculation Methods

Also provided herein is a method of generating a haploid sorghum embryo comprising performing emasculation on a first sorghum plant, wherein the emasculation comprises contacting the first plant with Trifluoromethanesulfonamide (also known as 1,1,1-Trifluoromethanesulfonamide, TFMSA, and CF₃SO₂NH₂) and crossing a male gamete derived from a haploid inducer plant with a female sorghum gamete derived from the first sorghum plant, thereby generating the haploid embryo. The emasculation can be performed before the crossing, to decrease the likelihood of the first sorghum plant being fertilized by self-produced pollen. For example, emasculation can be performed when the female plants are seedlings. Additionally and optionally, female flower parts on the first sorghum plant can be covered by bags during at least part of the method, again to decrease the likelihood of the first sorghum plant being fertilized by self-produced pollen or environmental pollen.

In some examples, contacting the first plant with TFMSA comprises applying TFMSA to one or more leaves of the first sorghum plant. Alternatively or additionally, TFMSA can be applied to the panicle or head of the first sorghum plant (e.g., the plant to be used as the female and the plant which is not the haploid inducer).

In some examples, the TFMSA can be applied 40-75 days after the first sorghum plant is planted. For example, the TFMSA can be applied 50-70 days after the first sorghum plant is planted or 55-65 days after the first sorghum plant is planted.

In some examples, the TFMSA can be applied to at least two leaves of the first sorghum plant. For example, the TFMSA can be applied to the newest fully developed leaf and the leaf on the next node above. In some examples, the TFMSA can be applied to at least 3 leaves or at least 4 leaves. Timing of the TFMSA application can be optimized or varied for specific sorghum varieties.

In some examples, the TFMSA is applied via an aqueous solution. In some examples, the TFMSA is applied via brushing, dropping, or spraying of the aqueous solution onto one or more leaves of the first sorghum plant. In some examples, the TFMSA is applied via brushing, dropping, or spraying of the aqueous solution onto one or more panicles or heads of the first sorghum plant.

In some examples, the aqueous solution can comprise the TFMSA at a concentration of 4 mM to 10 mM, inclusive. For example, the aqueous solution can comprise the TFMSA at a concentration ranging from 4 mM to 9 mM, 5 mM to 9 mM, 4 mM to 8 mM, 5 mM to 8 mM, 5 mM to 7 mM, 4 mM to 7 mM, or 6 mM to 7 mM. (The preceding ranges referring to TFMSA concentration include the endpoints).

In some examples, the aqueous solution comprises glycerol at a concentration of 2% to 10% by volume, inclusive. For example, the aqueous solution can comprise the glycerol at a concentration ranging from 2% to 9%, 2% to 8%, 2% to 7%, 2% to 6%, 2% to 5%, 3% to 9%, 3% to 8%, 3% to 7%, 3% to 6%, 3% to 5%, or 4% to 5%. (The preceding ranges referring to glycerol concentration include the endpoints and refer to percent by volume).

In some examples, the method can comprise obtaining a seed comprising the haploid embryo; confirming that the seed, or seedling resulting from the germination thereof, is haploid; and germinating the seed and contacting the haploid seedling with a chromosome doubling agent. In some examples, the method comprises confirming the haploid plant has become diploid.

Seed ploidy level can be confirmed by any method suitable to indicate chromosome number. For example, flow cytometry, genotyping of markers unique to the first female and haploid inducer plant, or a combination thereof can be used.

The haploid inducer plant can comprise any plant known to result in haploid embryos/seeds/plants when used to fertilize a female sorghum plant. In some examples, the haploid inducer plant can comprise a second sorghum plant such as, for example, the haploid inducer varieties disclosed herein (2PWCS25R, 2PNUM88R, or 2PCNW79R) or others such as SMHI01 and SMHI02. (See U.S. Pat. No. 11,089,748B2 and U.S. Pat. No. 11,737,405B2, which are incorporated herein, by reference).

In some examples, the haploid inducer plant comprises a Pennisetum plant such as, for example, a pearl millet plant (e.g., Cenchrus americanus also known as Pennisetum glaucum). (See, for example, US20230107598A1, which is incorporated by reference herein in its entirety).

All publications and patent applications mentioned in this specification are herein incorporated by reference to the same extent as if each individual publication or patent application was specifically and individually indicated to be incorporated by reference. Any definition(s) provided in this specification will control over any definition for the same word(s) provided in a document that has been incorporated by reference.

### CLAUSES OF THE DISCLOSURE

The present disclosure will be more completely understood by reference to the following numbered clauses:
1. A plant, non-seed plant part, seed, or cell of sorghum variety 2PWCS25R, representative seed of the variety having been deposited with the Nation Center for Marine Algae and Microbiota (NCMA) on Jul. 8, 2024 under accession number 202407001.
1A. A plant, non-seed plant part, seed, or cell of sorghum variety 2PWCS25R, seed of the variety having been deposited with the Nation Center for Marine Algae and Microbiota (NCMA) on Jul. 8, 2024 under accession number 202407001.
2. An sorghum haploid embryo or seed produced by crossing the plant of clause 1 or 1A with a second plant, wherein the second plant is used as a female.
3. A sorghum haploid plant produced by growing the sorghum haploid embryo, or seed containing the embryo, of clause 2.
4. The plant, non-seed plant part, seed, or plant cell of clause 1 or 1A, further comprising a locus conversion, wherein the plant or a plant grown from the plant part, seed, or plant cell otherwise comprises all of the physiological and morphological characteristics of sorghum variety 2PWCS25R when grown under the same environmental conditions.
5. A method of producing a sorghum haploid embryo or seed, the method comprising pollinating a female sorghum diploid plant with pollen from sorghum haploid inducer line 2PWCS25R, representative seed of the line having been deposited with the Nation Center for Marine Algae and Microbiota (NCMA) on Jul. 8, 2024 under accession number 202407001, wherein the pollination results in production of a sorghum haploid embryo and/or haploid seed.
5A. A method of producing a sorghum haploid embryo or seed, the method comprising pollinating a female sorghum diploid plant with pollen from sorghum haploid inducer line 2PWCS25R, seed of the line having been deposited with the Nation Center for Marine Algae and Microbiota (NCMA) on Jul. 8, 2024 under accession number 202407001, wherein the pollination results in production of a sorghum haploid embryo and/or haploid seed.
6. The method of clause 5 or 5A, further comprising, prior to pollinating the female sorghum diploid plant, performing emasculation by contacting the female diploid sorghum plant with Trifluoromethanesulfonamide (TFMSA).
7. The method of clause 5, 5A, or 6, further comprising contacting, with a chromosome doubling agent, any of (1) the sorghum haploid embryo, (2) haploid seed, or (3) a haploid plant germinated from the haploid seed or embryo.
8. The method of clause 7, further comprising producing a doubled haploid plant from the haploid embryo, seed, or plant and obtaining seed from the doubled haploid plant.
9. A sorghum seed produced by crossing the plant or non-seed plant part of clause 1 or 1A with a different plant.
10. An F1 plant produced by growing the seed of clause 9.
11. A plant part derived from the F1 plant of clause 10.
12. A method of producing a sorghum seed, the method comprising crossing two sorghum plants and harvesting the resultant sorghum seed, wherein at least one of the sorghum plants is the sorghum plant of clause 1 or 1A.
13. A method of producing a sorghum seed, the method comprising crossing two sorghum plants and harvesting the resultant sorghum seed, wherein at least one of the sorghum plants is the sorghum plant of clause 10.
14. A method of plant breeding, the method comprising: (a) crossing the plant or non-seed plant part of clause 1 or 1A with a second plant comprising a desired single locus to produce F1 progeny plant(s); and (b) selecting at least a first progeny plant from step (a) that comprises the single locus to produce a selected progeny plant.
15. A plant, non-seed plant part, seed, or cell of sorghum variety 2PNUM88R, representative seed of the variety having been deposited with the Nation Center for Marine Algae and Microbiota (NCMA) on Jul. 8, 2024 under accession number 202407002.
15A. A plant, non-seed plant part, seed, or cell of sorghum variety 2PNUM88R, seed of the variety having been deposited with the Nation Center for Marine Algae and Microbiota (NCMA) on Jul. 8, 2024 under accession number 202407002.
16. An F1 sorghum haploid embryo or seed produced by crossing the plant of clause 15 or 15A with a second plant, wherein the second plant is used as a female.
17. A sorghum haploid plant produced by growing the sorghum haploid embryo, or seed containing the embryo, of clause 16.
18. The plant, non-seed plant part, seed, or plant cell of clause 15 or 15A, further comprising a locus conversion, wherein the plant or a plant grown from the plant part, seed, or plant cell otherwise comprises all of the physiological and morphological characteristics of sorghum variety 2PNUM88R when grown under the same environmental conditions.
19. A method of producing a sorghum haploid embryo or seed, the method comprising pollinating a female sorghum diploid plant with pollen from sorghum haploid inducer line 2PNUM88R, representative seed of the line having been deposited with the Nation Center for Marine Algae and Microbiota (NCMA) on Jul. 8, 2024 under accession number 202407002, wherein the pollination results in production of a sorghum haploid embryo and/or haploid seed.
19A. A method of producing a sorghum haploid embryo or seed, the method comprising pollinating a female sorghum diploid plant with pollen from sorghum haploid inducer line 2PNUM88R, seed of the line having been deposited with the Nation Center for Marine Algae and Microbiota (NCMA) on Jul. 8, 2024 under accession number 202407002, wherein the pollination results in production of a sorghum haploid embryo and/or haploid seed.
20. The method of clause 19 or 19A, further comprising, prior to pollinating the female sorghum diploid plant, performing emasculation by contacting the female diploid sorghum plant with Trifluoromethanesulfonamide (TFMSA).
21. The method of clause 19, 19A, or 20, further comprising contacting, with a chromosome doubling agent, any of (1) the sorghum haploid embryo, (2) haploid seed, or (3) a haploid plant germinated from the haploid seed or embryo.
22. The method of clause 21, further comprising producing a doubled haploid plant from the haploid embryo, seed, or plant and obtaining seed from the doubled haploid plant.
23. A sorghum seed produced by crossing the plant or non-seed plant part of clause 15 or 15A with a different plant.
24. An F1 plant produced by growing the seed of clause 23.
25. A plant part derived from the F1 plant of clause 24.
26. A method of producing a sorghum seed, the method comprising crossing two sorghum plants and harvesting the resultant sorghum seed, wherein at least one of the sorghum plants is the sorghum plant of clause 15 or 15A.
27. A method of producing a sorghum seed, the method comprising crossing two sorghum plants and harvesting the resultant sorghum seed, wherein at least one of the sorghum plants is the sorghum plant of clause 24.
28. A method of plant breeding, the method comprising: (a) crossing the plant or non-seed plant part of clause 15 with a second plant comprising a desired single locus to produce F1 progeny plant(s); and (b) selecting at least a first progeny plant from step (a) that comprises the single locus to produce a selected progeny plant.
29. A plant, non-seed plant part, seed, or cell of sorghum variety 2PCNW79R, representative seed of the variety having been deposited with the Nation Center for Marine Algae and Microbiota (NCMA) on Jul. 8, 2024 under accession number 202407003.
29A. A plant, non-seed plant part, seed, or cell of sorghum variety 2PCNW79R, seed of the variety having been deposited with the Nation Center for Marine Algae and Microbiota (NCMA) on Jul. 8, 2024 under accession number 202407003.
30. An F1 sorghum haploid embryo or seed produced by crossing the plant of clause 29 or 29A with a second plant, wherein the second plant is used as a female.
31. A sorghum haploid plant produced by growing the sorghum haploid embryo, or seed containing the embryo, of clause 30.
32. The plant, non-seed plant part, seed, or plant cell of clause 29 or 29A, further comprising a locus conversion, wherein the plant or a plant grown from the plant part, seed, or plant cell otherwise comprises all of the physiological and morphological characteristics of sorghum variety 2PCNW79R when grown under the same environmental conditions.
33. A method of producing a sorghum haploid embryo or seed, the method comprising pollinating a female sorghum diploid plant with pollen from sorghum haploid inducer line 2PCNW79R, representative seed of the line having been deposited with the Nation Center for Marine Algae and Microbiota (NCMA) on Jul. 8, 2024 under accession number 202407003, wherein the pollination results in production of a sorghum haploid embryo and/or haploid seed.
33A. A method of producing a sorghum haploid embryo or seed, the method comprising pollinating a female sorghum diploid plant with pollen from sorghum haploid inducer line 2PCNW79R, seed of the line having been deposited with the Nation Center for Marine Algae and Microbiota (NCMA) on Jul. 8, 2024 under accession number 202407003, wherein the pollination results in production of a sorghum haploid embryo and/or haploid seed.
34. The method of clause 33 or 33A, further comprising, prior to pollinating the female sorghum diploid plant, performing emasculation by contacting the female diploid sorghum plant with Trifluoromethanesulfonamide (TFMSA).
35. The method of clause 33, 33A, or 34, further comprising contacting, with a chromosome doubling agent, any of (1) the sorghum haploid embryo, (2) haploid seed, or (3) a haploid plant germinated from the haploid seed or embryo.
36. The method of clause 37, further comprising producing a doubled haploid plant from the haploid embryo, seed, or plant and obtaining seed from the doubled haploid plant.
37. A sorghum seed produced by crossing the plant or non-seed plant part of clause 29 or 29A with a different plant.
38. An F1 plant produced by growing the seed of clause 37.
39. A plant part comprising at least one cell of the F1 plant of clause 38.
40. A method of producing a sorghum seed, the method comprising crossing two sorghum plants and harvesting the resultant sorghum seed, wherein at least one of the sorghum plants is the sorghum plant of clause 29 or 29A.
41. A method of producing a sorghum seed, the method comprising crossing two sorghum plants and harvesting the resultant sorghum seed, wherein at least one of the sorghum plants is the sorghum plant of clause 38.
42. A method of plant breeding, the method comprising: (a) crossing the plant or non-seed plant part of clause 29 or 29A with a second plant comprising a desired single locus to produce F1 progeny plant(s); and (b) selecting at least a first progeny plant from step (a) that comprises the single locus to produce a selected progeny plant.
43. A method of generating a haploid sorghum embryo comprising
   performing emasculation on a first sorghum plant, wherein the emasculation comprises contacting the first plant with Trifluoromethanesulfonamide (TFMSA) and
   crossing a male gamete derived from a haploid inducer plant with a female sorghum gamete derived from the first sorghum plant, thereby generating the haploid embryo.
44. The method of clause 43, wherein the contacting comprises applying TFMSA to one or more leaves of the first sorghum plant.
45. The method of clause 44, wherein the TFMSA is applied to at least two leaves of the first sorghum plant.
46. The method of any one of clauses 44-45, wherein the TFMSA is applied via an aqueous solution.
47. The method of any one of clauses 44-46, wherein the TFMSA is applied via brushing, dropping, or spraying of the aqueous solution onto one or more leaves of the first sorghum plant.
48. The method of any one of clauses 44-47, wherein the aqueous solution comprises the TFMSA at a concentration of 4 mM to 10 mM, inclusive.
49. The method of any one of clauses 46-48, wherein the aqueous solution further comprises glycerol at a concentration of 2% to 10% by volume, inclusive.
50. The method of any one of clauses 43-49, further comprising: obtaining a seed comprising the haploid embryo;
   confirming that the seed, or seedling resulting from the germination thereof, is haploid;
   germinating the seed and contacting the haploid seedling with a chromosome doubling agent; and
   confirming the haploid plant has become diploid.
51. The method of any one of clauses 43-50, wherein the haploid inducer plant comprises a second sorghum plant.
52. The method of any one of clauses 43-50, wherein the haploid inducer plant comprises a Pennisetum plant.
53. The method of clause 52, wherein the Pennisetum plant comprises a pearl millet plant.
54. A seed, plant, plant part, or plant cell of sorghum variety 2PWCS25R, sorghum variety 2PNUM88R, or sorghum variety 2PCNW79R, representative seed of the variety having been deposited under NCMA Accession Number 202407001, 202407002, or 202407003, respectively.
55. A plant, non-seed plant part, seed or plant cell of sorghum variety 2PWCS25R, sorghum variety 2PNUM88R, or sorghum variety 2PCNW79R, further comprising a locus conversion, wherein the plant or a plant grown from the plant part, seed or plant cell otherwise comprises all of the physiological and morphological characteristics of sorghum variety 2PWCS25R, sorghum variety 2PNUM88R, or sorghum variety 2PCNW79R, respectively, when grown under the same environmental conditions.
56. The plant, non-seed plant part, seed, or plant cell of clause 2, wherein the locus conversion confers a property selected from the group consisting of abiotic stress tolerance, altered phosphate content, altered antioxidant content, altered fatty acid profile, altered essential amino acid profile, altered carbohydrate content, herbicide resistance, insect resistance, disease resistance, and salt tolerance.
57. A method for producing a modified sorghum seed, plant, plant part, or plant cell, the method comprising applying plant breeding techniques to the seed, plant, plant part or plant cell of clause 54 to produce the modified sorghum seed, plant, plant part, or plant cell.
58. The method of clause 57, wherein applying plant breeding techniques comprises genome editing the seed, plant, plant part or plant cell to produce a genome-edited seed, plant, plant part, or plant cell and wherein the genome-edited seed, plant, plant part, or plant cell is modified by selfing, crossing, backcrossing, or a combination thereof to produce the modified sorghum seed, plant, plant part or plant cell.
59. The method of clause 57, wherein applying plant breeding techniques comprises selfing, crossing, backcrossing or a combination thereof the plant or plant part to produce a progeny seed, plant, plant part or plant cell and wherein the progeny seed, plant, plant part or plant cell is genome edited to produce the modified sorghum seed, plant, plant part or plant cell.
60. A sorghum seed, or an F1 plant grown therefrom, produced by crossing the plant or non-seed plant part of clause 54 with a different plant.
61. A plant part of clause 60, wherein the F1 plant is an F1 hybrid plant and wherein the plant part comprises at least one cell of the F1 hybrid plant.
62. A method of producing a sorghum seed, the method comprising crossing two sorghum plants and harvesting the resultant sorghum seed, wherein at least one of the sorghum plants is the sorghum plant of clause 54.
63. A sorghum seed produced by crossing the plant or non-seed plant part of clause 55 with a different plant.
64. An F1 hybrid plant produced by growing the seed of clause 63.
65. A plant part comprising at least one cell of the F1 hybrid plant of clause 64.
66. A non-seed plant part produced by growing the seed of clause 63.
67. A method of producing a sorghum seed, the method comprising crossing two sorghum plants and harvesting the resultant sorghum seed, wherein at least one of the sorghum plants is the sorghum plant of clause 55.
68. A method for producing a second sorghum plant or plant part, the method comprising doubling haploid seed generated from a cross of the plant or non-seed plant part of clause 55 with an inducer variety, thereby producing the second sorghum plant or plant part.
69. A method comprising isolating nucleic acids from the plant, non-seed plant part, seed or plant cell of clause 55.
70. A method for producing an F1 hybrid sorghum seed, the method comprising crossing an inbred plant grown from the modified sorghum seed, plant, plant part, or plant cell of clause 57 with a second inbred plant to produce the F1 hybrid sorghum seed.
71. A plant, non-seed plant part, seed, or plant cell of sorghum variety 2PWCS25R, sorghum variety 2PNUM88R, or sorghum variety 2PCNW79R, representative seed of the variety having been deposited under NCMA Accession Number 202407001, 202407002, or 202407003, respectively, the plant, non-seed plant part, seed, or plant cell further comprising a single locus conversion, wherein the single locus conversion is introduced into sorghum variety 2PWCS25R, sorghum variety 2PNUM88R, or sorghum variety 2PCNW79R by backcrossing or transformation.
72. The plant, plant part, seed, or plant cell of clause 71, wherein the locus conversion confers a property selected from the group consisting of abiotic stress tolerance, altered phosphate content, altered antioxidant content, altered fatty acid profile, altered essential amino acid profile, altered carbohydrate content, herbicide resistance, insect resistance, disease resistance, and salt tolerance.
73. The plant, plant part, seed, or plant cell of clause 71, wherein the locus conversion confers herbicide tolerance.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Screening for Haploid Inducer Lines

Screening of proprietary sorghum lines was performed to identify potential haploid inducers. The screening was preceded by crossing between haploid inducers as well as crossing between haploid inducers and non-haploid inducer sorghum varieties. The crossing included creation of F1, F2, F3, and backcrosses for several generations. Lines derived from all these crosses in different combinations and generations were evaluated for their ability to induce haploid progeny by either self-pollination or cross pollination to a female plant derived from a breeding cross. Lines with higher haploid induction rate were selected and moved forward to further selfing or back crossing. Self-crossed progenies of some of the promising haploid inducers were further self-crossed to generate homozygous populations of sorghum haploid inducers. 2PWCS25R, 2PNUM88R and 2PCNW79R were three lines with higher degrees of homozygosity and higher haploid induction rates. See Table 1.

Self-crossed haploid inducers were planted in an observation nursery and allowed to grow. Plants showing haploid phenotypes were selected for further determination of their ploidy status by flow cytometry/DNA markers. Haploid induction efficiencies were calculated based on the number of confirmed haploid plants within the nursery rows of a single entry.

To verify the ploidy level of putative haploid plants, leaf samples were taken from each plant, and each plant sample was tested using a standard flow cytometry protocol. Leaf samples of diploid plants were used as controls. Flow cytometry clearly distinguished haploid plants from diploid plants. In addition to this a set of 4000 DNA markers were analyzed for zygosity on DNA collected from putative haploid plants. A threshold of 50-60 markers were set as cut off for distinguishing haploids and diploids. For haploid inducer lines, a clear demarcation of haploid and diploid profiles was observed using the DNA markers. These data further supported the haploid phenotype of the selected putative haploid plants. See Table 1.

TABLE 1
Identification of 2PWCS25R, 2PNUM88R
and 2PCNW79R as haploid inducers

| | Haploid | Total | % |
|---|---|---|---|
| | plants | germinated | haploid |
| Genotype | obtained | plants | plants |
| 2PWCS25R | 4 | 78 | 5.13 |
| 2PNUM88R | 5 | 75 | 6.67 |
| 2PCNW79R | 3 | 35 | 8.57 |

### Example 2

### Determining Haploid Induction of 2PWCS25R on 11 Breeding Crosses

Maternal-haploid-inducing Sorghum line 2PWCS25R was used to pollinate 11 different female F1 plants (breeding crosses) which are shown in Table 2. Leaves of the female parent plants were treated with TFMSA to sterilize viable pollen. Panicles of the female plants were then pollinated with pollen collected from the head of the haploid inducer line. Seed was harvested when panicles were mature. Panicles harvested from the female plant contained seeds with haploid and diploid embryos. Randomly drawn sample from the pollinated female heads were planted in a field nursery. Leaf punches of plants with haploid phenotypes were collected from the field nursery and analyzed for DNA markers selected based on the DNA marker profile of the female line used in the cross. Haploids were identified based on the DNA marker profile. In another experiment, randomly selected 182 seeds from the heads collected from the female plant of the above experiment were subjected to DNA extraction and DNA marker analysis in a destructive manner. Number of haploid and diploid plants were identified based on the DNA marker profile. Table 2 shows the results of these two experiments. Column two first provides the haploid induction percentage from the field experiment and then the haploid induction percentage observed in the seed genotyping experiment. If a single value is provided in column two, the values were observed in only one experiment due to limited seed availability.

The different female genotypes were used in order to evaluate the effect with

different female backgrounds. The results indicate that each female line exhibits a varying degree of haploid frequencies when crossed with haploid inducer line 2PWCS25R.

TABLE 2
Haploid induction of 2PWCS25R on 11 different
female F1 plants (breeding crosses).

| Female F1 plant crossed | |
|---|---|
| with 2PWCS25R | HI % |
| Female plant 1 | 0; 0.54 |
| Female plant 2 | 0; 1.1 |
| Female plant 3 | 0; 0.5 |
| Female plant 4 | 0 |
| Female plant 5 | 0; 2.2 |
| Female plant 6 | 0 |
| Female plant 7 | 0; 1.6 |
| Female plant 8 | 0 |
| Female plant 9 | 1.6; 3.8 |
| Female plant 10 | 0 |
| Female plant 11 | 0; 1.1 |
| Self induction | 5.13 |

### Example 3

### Determining Haploid Induction Ability of an F1 Hybrid Between Haploid Inducer Lines

A maternal, haploid-inducing sorghum line derived from an F1 cross between SMHI01 and SMHI02 (“SMHI01/SMHI02 F1”) was used to pollinate 8 different female F1 plants (breeding crosses) shown in table 3. The head or panicles of the female parent plants were treated with TFMSA to sterilize viable pollen. Alternatively, hot water treatment or other emasculation methods can also be used for sterilizing viable pollen from the female parent plant. Panicles of the female plants were then pollinated with pollen collected from the head of the haploid inducer line. Seed was harvested when panicles were mature. Panicles harvested from the female plant contained seeds with haploid and diploid embryos. Randomly drawn samples from the pollinated female heads were planted in a field nursery. Leaf punches of plants with haploid phenotypes were collected from the field nursery and analyzed for DNA markers selected based on the DNA marker profile of the female line used in the cross. Haploids were identified based on the DNA marker profile. Table 3 shows results of haploid induction with the F1 cross between SMHI01/SMHI02. This experiment shows evidence that progeny of SMHI01 and SMHI02 can produce haploid inducers.

These results confirm that there is variability seen in the efficiency of haploid induction that is based on the female genotypes used in the experiment. Haploid induction efficiencies ranged from 0-4%, with one of the female genotypes matching the self-induction ability of the SMHI01/SMHI02 F1 hybrid.

TABLE 3
Haploid induction of SMHI01/SMHI02 F1 on
8 F1 female plants (breeding crosses)

| | Haploid induction rate |
|---|---|
| Cross | (%) |
| SMHI01/SMHI02 F1 × | 1 |
| female 1 | |
| SMHI01/SMHI02 F1 × | 0.67 |
| female 2 | |
| SMHI01/SMHI02 F1 × | 1.3 |
| female 3 | |
| SMHI01/SMHI02 F1 × | 0.3 |
| female 4 | |
| SMHI01/SMHI02 F1 × | 0.67 |
| female 5 | |
| SMHI01/SMHI02 F1 × | 0 |
| female 6 | |
| SMHI01/SMHI02 F1 × | 3.1 |
| female 7 | |
| SMHI01/SMHI02 F1 × | 4.02 |
| female 8 | |
| Self induction by | 4 |
| SMHI01/SMHI02 F1 | |

### Example 4

### Breeding Histories of 2PWCS25R, 2PNUM88R and 2PCNW79R

2PCNW79R was produced by crossing SMHI01 and SMHI02. The details of the breeding stages follow.

First cross SMHI01×SMHI02.

F1 was planted and crossed to SMHI02; seed was harvested in bulk.

BC1F1 was planted and backcrossed to SMHI02; seed was harvested in bulk.

BC2F1 was planted and harvested in bulk.

BC2F2 was planted and selected heads were harvested.

BC2F3 was planted and crossed to SMHI02.

BC3F1 was grown and harvested in bulk.

BC3F2 was grown and line was selected from individual heads. No further selection within the line was practiced.
BC3F2 selected line was grown and field evaluated.
BC3F2 selected line was coded.

Criteria used for selection were haploid induction rate and seed production.

Agronomics traits were considered as selection criteria during the process. Other selection criteria include: head type, plant height and plant type, uniformity and genetic purity.

2PNUM88R was produced by crossing SMHI01 and SMHI02. The details of the breeding stages follow.

First cross SMHI01×SMHI02.

F1 was planted, self pollinated and harvested in bulk.

F2 was grown and harvested in bulk.

F2 was grown and selected individual heads were crossed to SMHI02.

BC1F1 was grown and crossed to SMHI02. Harvest was in bulk.

BC2F1 was grown and crossed to SMHI02. Harvest was in bulk.

BC3F1 was grown and crossed to SMHI02. Harvest was in bulk.

BC3F2 seed was grown and line was selected from individual heads. No further selection within the line was practiced.

Criteria used for selection were haploid induction rate and seed production. Agronomics traits were considered as selection criteria during the process. Other selection criteria include: head type, plant height and plant type, uniformity and genetic purity.

2PWCS25R was produced by crossing SMHI01 and SMHI02. The details of the breeding stages follow.

First cross SMHI02 x SMHI01.

F1 was planted, self pollinated and harvested in bulk.

F2 was planted and individual plant crossed to SMHI02 to make BC1 seed.

BC1F1 seed was grown and individual plants were selected, self pollinated and harvest individually.

BC1F2 was grown and harvested in bulk.

BC1F3 was grown and harvested in bulk.

BC1F3 was planted and backcrossed to SMHI02.

BC2F1 was grown and field evaluated.

BC2F1 was selected and line was coded from selected plants. No further selection within the line was practiced.

Criteria used for selection were haploid induction rate and seed production. Agronomics traits were considered as selection criteria during the process. Other selection criteria include: head type, plant height and plant type, uniformity and genetic purity.

### DEPOSITS

Applicant has made a deposit of 625 seeds of Sorghum Line 2PWCS25R with the NCMA, 60 Bigelow Drive, East Boothbay, ME 04544, USA. The seeds deposited with the NCMA on Jul. 8, 2024 were obtained from the seed of the variety maintained by Pioneer Hi-Bred International, Inc., 7250 NW 62nd Avenue, Johnston, Iowa, 50131 since prior to the filing date of this application. Access to this seed will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. § 1.808. This deposit of the Sorghum Line 2PWCS25R will be maintained in the NCMA depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. §§ 1.801-1.809, including providing an indication of the viability of the sample upon deposit. Applicant has no authority to waive any restrictions imposed by law on the transfer of biological material or its transportation in commerce. Applicant does not waive any infringement of rights granted under this patent or under the Plant Variety Protection Act (7 USC 2321 et seq.). The deposit is recorded under accession number 202407001.

Applicant has also made a deposit of 625 seeds of Sorghum Line 2PNUM88R with the NCMA, 60 Bigelow Drive, East Boothbay, ME 04544, USA. The seeds deposited with the NCMA on Jul. 8, 2024 were obtained from the seed of the variety maintained by Pioneer Hi-Bred International, Inc., 7250 NW 62nd Avenue, Johnston, Iowa, 50131 since prior to the filing date of this application. Access to this seed will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. § 1.808. This deposit of the Sorghum Line 2PNUM88R will be maintained in the NCMA depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. §§ 1.801-1.809, including providing an indication of the viability of the sample upon deposit. Applicant has no authority to waive any restrictions imposed by law on the transfer of biological material or its transportation in commerce. Applicant does not waive any infringement of rights granted under this patent or under the

Plant Variety Protection Act (7 USC 2321 et seq.). The deposit is recorded under accession number 202407002.

Applicant has also made a deposit of 625 seeds of Sorghum Line 2PCNW79R with the NCMA, 60 Bigelow Drive, East Boothbay, ME 04544, USA. The seeds deposited with the NCMA on Jul. 8, 2024 were obtained from the seed of the variety maintained by Pioneer Hi-Bred International, Inc., 7250 NW 62nd Avenue, Johnston, Iowa, 50131 since prior to the filing date of this application. Access to this seed will be available during the pendency of the application to the Commissioner of Patents and Trademarks and persons determined by the Commissioner to be entitled thereto upon request. Upon allowance of any claims in the application, the Applicant will make the deposit available to the public pursuant to 37 C.F.R. § 1.808. This deposit of the Sorghum Line 2PCNW79R will be maintained in the NCMA depository, which is a public depository, for a period of 30 years, or 5 years after the most recent request, or for the enforceable life of the patent, whichever is longer, and will be replaced if it becomes nonviable during that period. Additionally, Applicant has or will satisfy all of the requirements of 37 C.F.R. 881.801-1.809, including providing an indication of the viability of the sample upon deposit. Applicant has no authority to waive any restrictions imposed by law on the transfer of biological material or its transportation in commerce. Applicant does not waive any infringement of rights granted under this patent or under the Plant Variety Protection Act (7 USC 2321 et seq.). The deposit is recorded under accession number 202407003.

What is claimed is:

1. A plant, non-seed plant part, seed, or cell of sorghum variety 2PWCS25R, sorghum variety 2PNUM88R, or sorghum variety 2PCNW79R, representative seed of the variety having been deposited with the Nation Center for Marine Algae and Microbiota (NCMA) under accession number 202407001, 202407002, or 202407003, respectively.

2. An F1 sorghum haploid embryo or seed produced by crossing the plant of claim 1 with a second plant, wherein the second plant is used as a female.

3. A sorghum haploid plant produced by growing the sorghum haploid embryo, or seed containing the embryo, of claim 2.

4. The plant, non-seed plant part, seed, or plant cell of claim 1, further comprising a locus conversion, wherein the plant or a plant grown from the plant part, seed, or plant cell otherwise comprises all of the physiological and morphological characteristics of sorghum variety 2PWCS25R when grown under the same environmental conditions.

5. A sorghum seed produced by crossing the plant or non-seed plant part of claim 1 with a different plant.

6. An F1 plant produced by growing the seed of claim 5.

7. A plant part derived from the F1 plant of claim 6.

8. A method of producing a sorghum seed, the method comprising crossing two sorghum plants and harvesting the resultant sorghum seed, wherein at least one of the sorghum plants is the sorghum plant of claim 1.

9. A method of producing a sorghum seed, the method comprising crossing two sorghum plants and harvesting the resultant sorghum seed, wherein at least one of the sorghum plants is the sorghum plant of claim 6.

10. A method of plant breeding, the method comprising: (a) crossing the plant or non-seed plant part of claim 1 with a second plant comprising a desired single locus to produce F1 progeny plant(s); and (b) selecting at least a first progeny plant from step (a) that comprises the single locus to produce a selected progeny plant.

11. A method of producing a sorghum haploid embryo or seed, the method comprising pollinating a female sorghum diploid plant with pollen from sorghum haploid inducer line 2PWCS25R, sorghum haploid inducer line 2PNUM88R, or sorghum haploid inducer line 2PCNW79R, representative seed of the line having been deposited with the Nation Center for Marine Algae and Microbiota (NCMA) under accession number 202407001, 202407002, or 202407003, respectively, wherein the pollination results in production of a sorghum haploid embryo and/or haploid seed.

12. The method of claim 11, further comprising, prior to pollinating the female sorghum diploid plant, performing emasculation by contacting the female diploid sorghum plant with Trifluoromethanesulfonamide (TFMSA).

13. The method of claim 11, further comprising contacting, with a chromosome doubling agent, any of (1) the sorghum haploid embryo, (2) haploid seed, or (3) a haploid plant germinated from the haploid seed or embryo.

14. The method of claim 13, further comprising producing a doubled haploid plant from the haploid embryo, seed, or plant and obtaining seed from the doubled haploid plant.

15. A method of generating a haploid sorghum embryo comprising
performing emasculation on a first sorghum plant, wherein the emasculation comprises contacting the first plant with Trifluoromethanesulfonamide (TFMSA) and
crossing a male gamete derived from a haploid inducer plant with a female sorghum gamete derived from the first sorghum plant, thereby generating the haploid embryo.

16. The method of claim 15, wherein the contacting comprises applying TFMSA to one or more leaves of the first sorghum plant.

17. The method of claim 16, wherein the TFMSA is applied to at least two leaves of the first sorghum plant.

18. The method of claim 16, wherein the TFMSA is applied via an aqueous solution.

19. The method of claim 16, wherein the TFMSA is applied via brushing, dropping, or spraying of the aqueous solution onto one or more leaves of the first sorghum plant.

20. The method of claim 16, wherein the aqueous solution comprises the TFMSA at a concentration of 4 mM to 10 mM, inclusive.
